(19) United States
(12) Patent Application Publication
(10) Pub. No.: US 2012/0109043 A1
(43) Pub. Date: May 3, 2012

(54) Integrated skin-treatment specimen dispenser

(76) Inventors: Yuchen Zhou, San Jose, CA (US); Hieu Tieu, San Jose, CA (US); Chao Uei Wahng, Fremont, CA (US)

(21) Appl. No.: 13/317,136

(22) Filed: Oct. 11, 2011

Related U.S. Application Data
(60) Provisional application No. 61/456,164, filed on Nov. 2, 2010.

(51) Int. Cl. A61M 35/00

(52) U.S. Cl. 604/20; 604/22

(57) ABSTRACT

A device for skin treatment by concurrently applying a series of physical movement such as ultrasound vibration and dispensing a specimen to a target kin area is disclosed. The device includes a skin treatment surface for treating the target skin area, a cartridge containing specimen to be dispensed to the target skin area, an ultrasound generator, a control unit and a power supply. A specimen outlet is on the kin treatment plate. The outlet is coupled to the cartridge.

### REFERENCE TO RELATED APPLICATION

The present application claims priority to the provisional Appl. Ser. No. 61/456,164 filed on Nov. 2, 2010, the entire content of which is hereby incorporated by reference.

### FIELD OF THE INVENTION

The present invention relates generally to ultrasonic technology. More particularly, it relates to a novel ultrasonic skin care device with a built-in integrated skin-treatment specimen dispenser along with an electrical interface to enable easy application of the specimen for hygiene operation and skin beautification purposes.

### BACKGROUND OF THE INVENTION

It has been well realized that ultrasonic technologies can be used in skin treatment and beautification. FIG. 1 is a schematic diagram shows illustrating a typical ultrasonic skin treatment device, the structure of which is commonly shared by all available products in the market. The device contains a device body 1, i.e. a handle or a casing, where resides electronics that controls an ultrasound transducer 3. The device body 1 may have other functions such as power control and charging which are not shown in FIG. 1. The device contains a vibration plate 2, which is usually metallic, that contacts human skin with its external surface 4 and is driven by the ultrasound transducer 3 which is coupled to the internal surface of the plate 2. The vibration at ultrasonic frequency from the transducer 3 is transmitted to the human skin or skin treatment products via the external surface 4. For easy handling, the plate 2 and the transducer 3 are enclosed in a housing, which is not shown in FIG. 1, of the device body 1.

In prior art devices, due to the design feature as FIG. 1 shows, skin treatment specimen is dispensed from a separate specimen container either directly to the skin surface or to the external surface 4 of the plate 2 before treatment on target skin area with simultaneous application of the ultrasound vibration from the plate 2. This process of application of skin treatment specimen from an external container limits the device's portability, where the specimen application from separate container not only increases the complexity of the skin treatment process but also imposes hygiene concerns if such skin treatment process is not to be performed in a dedicated area such as a beauty spa or a private room, but in a public area with frequent traffic such as office, public transportation or elevator. For daily beautification purpose, it is desired that such ultrasound treatment process can be extended into a typical user's day time, other than the usual limited time skin treatment process in the morning or at night, in order to benefit from the full scale that ultrasonic treatment can offer. Prior arts due to their limited portability and need of separate specimen containment, can not meet the stated treatment requirement because of the complexity of the application process.

What is desired is a new design ultrasonic device for generating ultrasound vibration and applying specimen concurrently, which is extremely portable and can relieve the user from the external specimen application process such that ultrasound treatment can be easily done at the time periods that are not usually feasible for using prior art designs.

### SUMMARY OF THE INVENTION

The present application discloses and teaches a novel solution of utilizing an integrated specimen dispenser with electrical interface in ultrasonic skin treatment device and other electronic skin treatment devices. Methods to utilize the electrical interface to achieve easier and better skin beautification process are also disclosed.

It is an object of this invention to integrate a skin treatment specimen dispenser having an electrical interface with an ultrasonic vibration generation device to produce an ultra portable and hygiene solution to enable extended ultrasound treatment for daily skin beautification.

It is another object of this invention to integrate a skin treatment specimen dispenser having an electrical interface with an electronic skin treatment device.

Yet it is another object of this invention to provide possible methods to enhance portability, flexibility and customizability of skin treatment devices with integrated specimen dispenser having an electrical interface.

In a first embodiment of this invention, we described a skin-treatment specimen dispenser integrated within an ultrasonic massaging device, wherein the dispenser has an electrical interface that communicates with the control electronics of the massaging device.

We have also realized that the enhanced portability, flexibility and customizability by an integrated specimen dispenser with electrical interface can not only benefit ultrasound based skin treatment devices, but also benefit all electronic skin treatment devices that operate by acting on human skin with a physical touching such as vibration, brushing, abrasion, piercing, and optical lightening, etc. and with an externally applied skin treatment specimen such as liquid, gel, serum, cream, lotion, paste and powder.

In a second embodiment of this invention, we described a skin-treatment specimen dispenser integrated with an electronic skin treatment device, where the dispenser has an electrical interface that communicates with the control electronics of the skin-treatment device.

### BRIEF DESCRIPTION OF THE DRAWINGS

FIG. 1 is a schematic diagram illustrating a typical ultrasound skin care design according to the prior arts;

FIG. 2A is a schematic diagram illustrating a front view of an ultrasonic device with an integrated specimen dispenser according to the first preferred embodiment of the present invention;

FIG. 2B is a schematic diagram illustrating a cross-sectional view of the specimen dispenser according to FIG. 2A;

FIG. 2C is a schematic diagram illustrating a perspective view of the specimen cartridge according to FIG. 2A and FIG. 2B;

FIG. 3A is a schematic diagram illustrating a front view of an ultrasonic device with an integrated specimen dispenser according to the second preferred embodiment of the present invention;

FIG. 3B is a schematic diagram illustrating a cross-sectional view of the specimen dispenser according to FIG. 3A;

FIG. 3C is a schematic diagram illustrating a perspective view of the specimen cartridge according to FIG. 3A and FIG. 3B;

FIG. 4A is a schematic diagram illustrating a perspective view of a typical exterior shape of the device according to the present invention;

FIG. 4B is a schematic diagram illustrating a perspective view of the device and a contactless charger frame; and

FIG. 4C is a schematic diagram illustrating a side view of the device and the contactless charger frame of FIG. 4B.

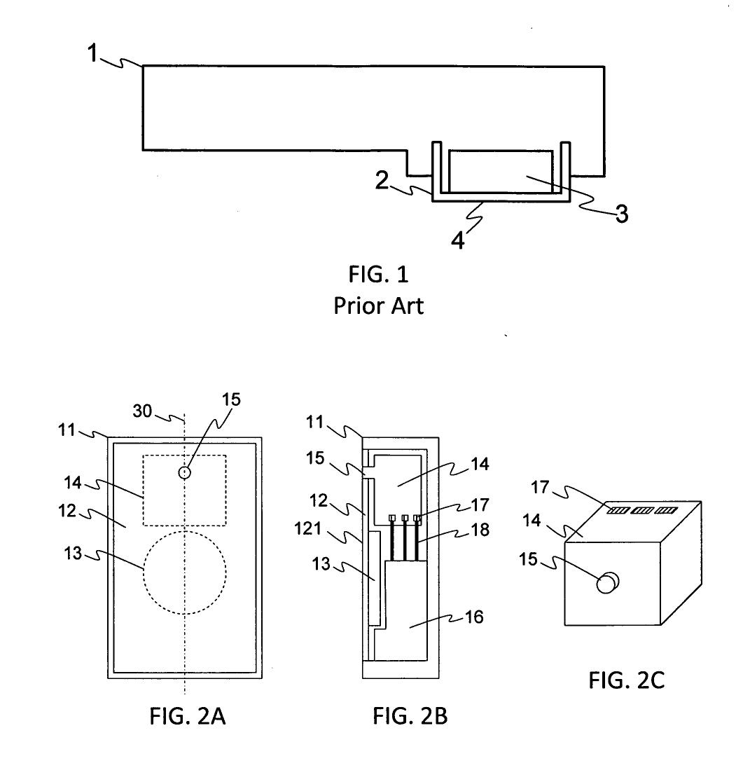

### DETAILED DESCRIPTION OF THE INVENTION

While the present invention may be embodied in many different shapes, forms, designs or configurations, for the purpose of promoting an understanding of the principles of the invention, reference will be made to the embodiments illustrated in the drawings and specific language will be used to describe the same. It will nevertheless be understood that no limitation of the scope of the invention is thereby intended. Any alterations and further implementations of the principle, the essence or the spirit of the invention as described herein are contemplated as would normally occur to one skilled in the art to which the invention relates.

Please note that although the first preferred embodiment is described first in this document, the invention described as the second preferred embodiment is generic to the first preferred embodiment. In other words, the technology described as the first preferred embodiment is a specific application of the first embodiment.

### First Preferred Embodiment

FIG. 2A illustrates a front view of the proposed device according to the first preferred embodiment of this invention. FIG. 2B illustrates a cross-sectional view along the center line 30 of FIG. 2A. FIG. 2C illustrates a perspective view of the specimen cartridge 14 which can be installed in and taken out from the device body.

The device according to the first preferred embodiment contains following aspects: (1) an enclosure body 11, which can be in any three dimensional shape, such as cube, cuboid, globe, ellipsoid, cylinder or irregular shape; (2) an ultrasound transmission plate 12 for contacting the skin and transmitting ultrasonic vibration generated by an ultrasound generator 13 to a target skin area; (3) a skin treatment specimen container and dispenser, i.e. the specimen cartridge 14; (4) a specimen outlet 15 existing on the same continuous surface of the device as the transmission plate 12, through which skin treatment specimen is dispensed close to or, preferably, directly on top of the transmission plate 12 surface 121 that is to be in contact with the skin during skin treatment; (5) one or more electrical contacts 17 that can be electrically accessed by an electrical interface 18; (6) the electrical interface 18 that electrically couples the cartridge 14 and a control unit 16 together; (7) the control unit 16 that contains electrical circuits and various semiconductor components, which controls ultrasonic vibration generation from the ultrasound generator 13, provides user interface, power supply and charging functions. Additionally, the control unit 16 sends electrical signals to the specimen cartridge 14 or receives electrical signals from the specimen cartridge 14 to achieve required skin treatment procedure. Note that the ultrasound generator 13 is enclosed in the device's body. It is mechanically coupled to the ultrasound transmission plate 12 and electrically coupled to the control unit 16.

In the most preferred mode, the enclosure body 11 is in an easy-holding oval shape and includes two continuous pieces—front and back pieces—which are mechanically coupled together. The specimen outlet 15 is on the front piece immediately coupled to the ultrasonic transmission plate 12. In use, the back piece is for palm-holding. The device includes a wireless or contactless charger. Without electrical wire contact, the device can be charged with electrical power from a charger base connected with power supply. So, except the specimen outlet 15, the device does not have any other outlet or connectors. The outlet 15 is typically a small round hole with a diameter of less than 1 mm.

The skin treatment specimen contained in the cartridge 14 can be, but not limited to, liquid, gel, serum, cream, lotion, paste and powder.

The structure of the cartridge 14 may have any one or more of the following features: (1) it is removable and replaceable, i.e. it may be taken out and installed back into the enclosure body 11 by the user; (2) it is reusable, i.e., the specimen may be replenished within the cartridge 14 by the user after depletion of the specimen during skin beatification process; (3) it is disposable and for one-time use only, where specimen is pre-filled within the cartridge before usage; (4) it can be configured as an integrated cartridge with multiple cartridges containing same or different specimens and the cartridges can be individually selected to dispense contained specimen; and (5) it can be configured as an integrated cartridge having multiple specimen compartments that may contain same or different specimens and each compartment within the cartridge can be individually selected and dispense specimen.

The configuration between and among the cartridge 14, the electrical contact 17 and the control unit 16 may have any one or more of the features of: (1) the electrical contact 17 can be a single contact point or an array of contacts; (2) the embedded specimen dispensing or releasing mechanism within the cartridge 14 is controlled by the control unit 16 through the electrical interface 18 and the contact 17; (3) the cartridge 14 may comprise an embedded memory or data storage device for storing operation data; (4) the control unit 16 may receive data stored in the cartridge 14 to display information to the user through visual, skin contact or sound effects; (5) the control unit 16 may receive data stored in the cartridge 14 to operate the ultrasonic generator 13 in a specific manner determined by the information stored in the memory of the cartridge 14; (6) the control unit 16 may comprise an embedded memory or data storage device for storing information such as, but not limited to, operation data, user skin condition data, user personal information, and cartridge identification data; (7) the control unit 16 may send data to be stored in the cartridge 14; and (8) the system setting of the cartridge 14 may be recovered by the manufacturer and the data pre-stored within the cartridge 14 may be retrieved.

The data stored in the embedded memories or data storage devices are in digital format and may include any of: (1) information of the specimen contained within the cartridge, such as but not limited to, specimen brand, name, type, origin, composition, production date and expiration date, specimen level within the cartridge and ordering information; (2) information of optimal or pre-set operational mode of the ultrasonic generator 13 through the control unit 16 when the specimen contained in the cartridge 14 is to be used, such operational mode can be, but not limited to, timing, ultrasonic vibration strength and location of the operation to be generated on transmission plate 12; (3) information of optimal or pre-set operational mode of the different cartridges 14 or difference specimen compartments within a single cartridge, such operational mode can be, but not limited to, timing of specimen application from each different cartridge or each different compartment, amount of specimen to be dispensed from each different cartridge or each different compartment; (4) information of historic usage data of the device, the cartridge and specimen used; (5) information created or input by the user; and, (6) biographic information of the user.

### Second Preferred Embodiment

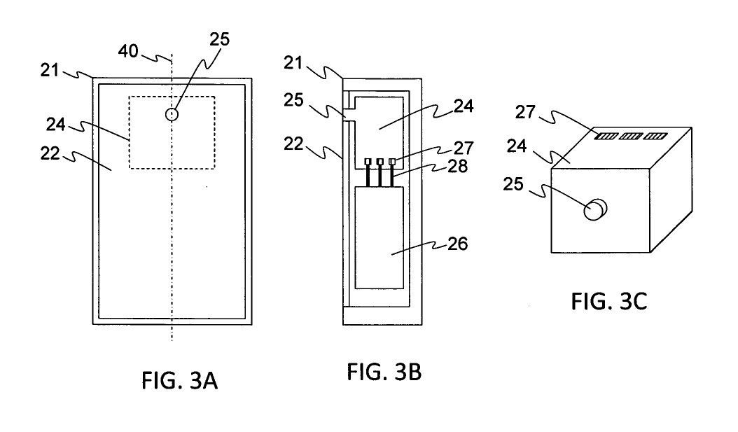

FIG. 3A illustrates a front view of the device according to the second preferred embodiment of this invention. FIG. 2B illustrates a cross-sectional view along the center line 40 of FIG. 3A. FIG. 3C illustrates a perspective view of the specimen cartridge 24.

The device according to the second preferred embodiment contains following aspects: (1) an enclosure body 21; (2) a skin treatment side 22 for treating a target skin area with a physical means; (3) a specimen cartridge 24, which contains skin treatment specimen such as, but not limited to, liquid, gel, serum, cream, lotion, paste and powder; (3) a specimen outlet 25 existing on the skin treatment side 22, through which skin treatment specimen is dispensed close to or, preferably, directly on the area of the skin treatment side 22 that is to be in contact with the skin during skin treatment; (4) one or more electrical contacts 27 that can be electrically accessed by an electrical interface 28; (5) the electrical interface 28 that electrically connects or couples the cartridge 24 and a control unit 26; (6) the control unit 26 that contains electrical circuits and semiconductor components, which controls skin treatment process by the skin treatment side 22, provides user interface, power supply and charging functions, and sends electrical signals to the specimen cartridge 24 or receives electrical signals from the specimen cartridge 24 to achieve required skin treatment procedure.

The treatment side 22 can be, but not limited to, a vibrating surface, a rotating brush, a moving or stationary surface with macro (size>1 mm) or micro (size<1 mm) protruding structures, a single or an array of light emitting elements such as LEDs. The physical means can be, vibration, brushing, abrasion, piercing, and optical lightening.

The structure of the cartridge 24 may have any one or more of the following features: (1) it is removable and replaceable, i.e. it may be taken out and installed back into the enclosure body 21 by the user; (2) it is reusable, i.e., the specimen may be replenished within the cartridge 24 by the user after depletion of the specimen during skin beatification process; (3) it is disposable and for one-time use only, where specimen is pre-filled within the cartridge before usage; (4) it can be configured as an integrated cartridge with multiple cartridges containing same or different specimens and the cartridges can be individually selected to dispense contained specimen; and (5) it can be configured as an integrated cartridge having multiple specimen compartments that may contain same or different specimens and each compartment within the cartridge can be individually selected and dispense specimen.

The configuration between and among the cartridge 24, the electrical contact 27 and the control unit 26 may have any one or more of the features of: (1) the electrical contact 27 can be a single contact point or an array of contacts; (2) the embedded specimen dispensing or releasing mechanism within the cartridge 24 is controlled by the control unit 26 through the electrical interface 28 and the contact 27; (3) the cartridge 24 comprises an embedded memory or data storage device for storing operation data; (4) the control unit 26 may receive data stored in the cartridge 24 to display information to the user through visual, skin contact or sound effects; (5) the control unit 26 may receive data stored in the cartridge 24 to operate the treatment side 22 in a specific manner determined by the information stored in the memory of the cartridge 24; (6) the control unit 26 may comprise an embedded memory or data storage device for storing information such as, but not limited to, operation data, user skin condition data, user personal information, and cartridge identification data; (7) the control unit 26 may send data to be stored in the cartridge 24; and (8) the system setting of the cartridge 24 may be recovered by the manufacturer and the data pre-stored within the cartridge 24 may be retrieved.

The data stored the embedded memories or data storage devices are in digital format and may include any of: (1) information of the specimen contained within the cartridge, such as but not limited to, specimen brand, name, type, origin, composition, production date and expiration date, specimen level within the cartridge and ordering information; (2) information of optimal or pre-set operational mode of the treatment side 22 through the control unit 26 when the specimen contained in the cartridge 24 is to be used, such operational mode can be, but not limited to, timing, the strength of the physical means applied to the skin and location of the physical means to be generated by the treatment side 22 on the skin; (3) information of optimal or pre-set operational mode of the different cartridges 24 or difference specimen compartments within a single cartridge, such operational mode can be, but not limited to, timing of specimen application from each different cartridge or each different compartment, amount of specimen to be dispensed from each different cartridge or each different compartment; (4) information of historic usage data of the device, the cartridge and specimen used; (5) information created or input by the user; and (6) biographic information of the user.

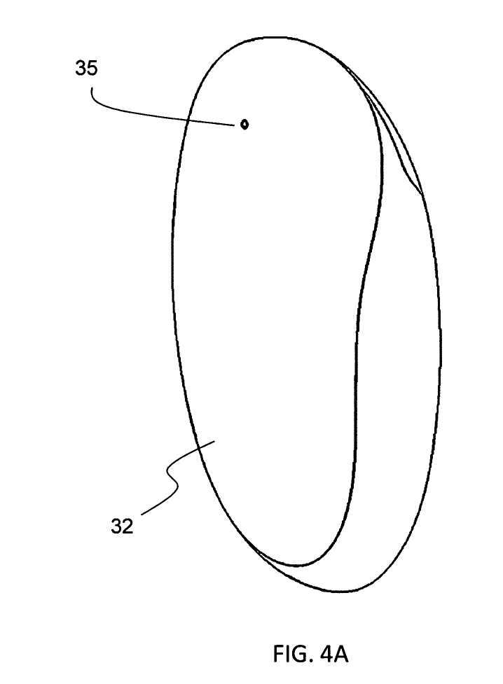

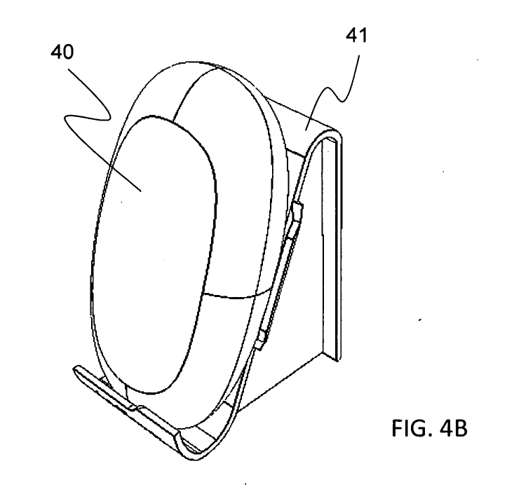

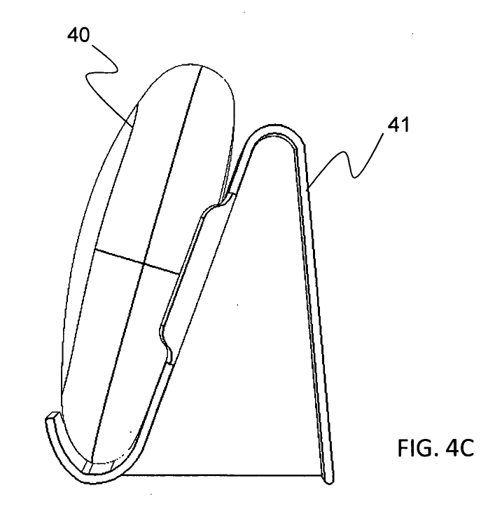

The device according to the present invention has a symmetrical body with smooth exterior surface. Preferably, the body is in an ellipsoidal shape. The body has a first member and a second member mechanically coupled together. The first member is the skin treatment plate. Except that the first member has a specimen outlet, the first member and the second member's exterior shapes are identical or substantially same. As an example, FIG. 4 illustrates a typical exterior shape of the device according to the present invention, wherein it contains a skin treatment side 32 and a specimen outlet 35 on said treatment side 32. The outlet 35 is typically a small round hole with a diameter of less than 1 mm.

FIG. 4B is a schematic diagram illustrating a perspective view of the device 40 and a contactless charger frame 41. The charger frame 41 is coupled to the power supply. When the device 40 is properly placed in the frame, the battery in the device 40 is wireless charged. FIG. 4C is a schematic diagram illustrating a side view of the device 40 and the contactless charger frame 41 of FIG. 4B.

Advantages of this invention are numerous, for examples: (1) integrated specimen dispenser with electrical interface enhances the portability, flexibility and customizability of the skin treatment process for both ultrasonic skin treatment devices and other electronic skin treatment devices; and (2) embedded memory within the cartridge containing product information, best mode of operation, pre-set beautification process and usage data greatly increases the positive effect of the skin beautification process, reduces the complexity of the user's operation and provides means of feedback from user to manufacture for further improvement on the skin care products

While one or more embodiments of the present invention have been illustrated above, the skilled artisan will appreciate that modifications and adoptions to those embodiments may be made without departing from the scope and spirit of the present invention.

1. A device for skin treatment by concurrently applying a series of physical movement and dispensing a specimen to a target skin area, comprising:
a skin treatment member for treating the target skin area with a series of physical movement; and
a specimen outlet on said skin treatment member, said outlet coupled to a cartridge containing specimen to be dispensed to the target skin area;
wherein said cartridge is electrically coupled to a control unit through an electrical interface and one or more electrical contacts; wherein said control unit comprises a user interface and a number of electrical circuits and semiconductor components which collectively control said treatment member's physical movement and specimen dispense from said cartridge; wherein said control unit is electrically coupled to a power supply; wherein said cartridge, said electrical interface, said control unit and said power supply are all enclosed in a device body of said device.

2. The device of claim 1, said a specimen is any of: liquid, gel, serum, cream, lotion, paste and powder.

3. The device of claim 1, wherein said cartridge is any of: a removable and replaceable cartridge; a refillable cartridge; a disposable and for one-time use only cartridge; an integrated cartridge having multiple sub-cartridges containing same or different specimens, said sub-cartridges being individually selected to dispense specimen therein; and an integrated cartridge with multiple specimen compartments containing same or different specimens, each of said compartments being individually selected to dispense specimen therein.

4. The device of claim 1, wherein said electrical interface is any of: a single contact point; and an array of contacts.

5. The device of claim 1, wherein said cartridge comprises an embedded data storage device for storing operational data; wherein said control unit is capable of receiving data stored in said cartridge to display information to a user through visual, skin contact or sound effects and is capable of receiving data stored in said cartridge to operate said skin treatment member in a specific manner determined by data stored in said storage device; wherein said control unit is capable of sending data to be stored in said cartridge; and wherein data stored in said cartridge is retrievable.

6. The device of claim 1, wherein said control unit comprises an embedded data storage device to store operational information for defining said skin treatment member's operation manner, and wherein said operational information is any of:
operation data; user skin condition data; user personal information; and cartridge identification data.

7. The device of claim 6, wherein said control unit is capable of displaying said information to a user through visual, skin contact or sound effects.

8. The device of claim 1, wherein said skin treatment member is any of: a vibrating surface, a rotating brush, a moving surface with protruding structures, a stationary surface with protruding structures, a single light and an array of light emitting elements.

9. The device of claim 1, further comprising an ultrasound generator which is mechanically coupled to said skin treatment member and electrically coupled to said control unit.

10. The device of claim 1, further comprising a contactless charger which is electrically coupled to said power supply.

11. The device of claim 1, wherein said device's body consists of a first piece and a second piece mechanically coupled together, wherein said first piece is said skin treatment member and said second piece's exterior shape is symmetrical to said first piece's exterior shape.

12. The device of claim 1, wherein said device's is in any shape of: cube, cuboid, globe, ellipsoid, cylinder or an irregular shape.

13. A device for skin treatment by concurrently applying ultrasound vibration and dispensing a specimen to a target skin area, comprising:
a body in an ellipsoidal shape with smooth exterior surface, said body comprising a first member and a second member mechanically coupled together, and said first member's and second member's shapes being symmetrical;
an ultrasound generator mechanically coupled to said first member;
a specimen outlet on said first member, said outlet coupled to a cartridge containing specimen to be dispensed to the target skin area;
wherein said cartridge and said ultrasound generator are electrically coupled to a control unit through an electrical interface and one or more electrical contacts; wherein said control unit comprises a user interface and a number of electrical circuits and semiconductor components which collectively control said ultrasound generator's output and said cartridge's dispense of specimen; wherein said control unit, said ultrasound generator and cartridge are electrically coupled to a power supply which is coupled to a charger; wherein said cartridge, said ultrasound generator, said electrical interface, said control unit, said power supply and said charger are all located inside of said device's body.

14. The device of claim 13, said a specimen is any of: liquid, gel, serum, cream, lotion, paste and powder.

15. The device of claim 13, wherein said cartridge comprises multiple sub-cartridges containing same or different specimens and said sub-cartridges can be individually selected to dispense specimen therein.

16. The device of claim 13, wherein said cartridge comprises multiple specimen compartments containing same or different specimens and each of said compartments can be individually selected to dispense specimen therein.

17. The device of claim 13, wherein said cartridge comprises an embedded data storage device for storing operational data; wherein said control unit is capable of receiving data stored in said cartridge to display information to a user through visual, skin contact or sound effects and is capable of receiving data stored in said cartridge to operate said first member in a specific manner determined by data stored in said storage device; wherein said control unit is capable of sending data to be stored in said cartridge; and wherein data stored in said cartridge is retrievable.

18. The device of claim 13, wherein said control unit comprises an embedded data storage device for storing operational information; and wherein said control unit is capable of displaying said information to a user through visual, skin contact or sound effects and is capable of operating said skin treatment member in a specific manner determined by data stored in said storage device.

19. The device of claim 18, wherein said operational information can be, but not limited to, operation data, user skin condition data, user personal information, and cartridge identification data.

20. The device of claim 13, wherein said charger is a contactless charger.
